(19) United States
(12) Patent Application Publication
(10) Pub. No.: US 2024/0156137 A1
(43) Pub. Date: May 16, 2024

(54) COMPOSITIONS AND METHODS FOR PRODUCING NON-HUMAN TISSUE ENGINEERED MEAT PRODUCTS

(71) Applicant: MYOS CORP., Cedar Knolls, NJ (US)

(72) Inventors: Joseph MANNELLO, Cedar Knolls, NJ (US); Neerav D. PADLIYA, Cedar Knolls, NJ (US)

(21) Appl. No.: 18/502,249

(22) Filed: Nov. 6, 2023

Related U.S. Application Data
(60) Provisional application No. 63/425,513, filed on Nov. 15, 2022.

(51) Int. Cl. A23L 13/00; C12N 5/077

(52) U.S. Cl. CPC A23L 13/00; C12N 5/0658; C12N 2500/84; C12N 2513/00

(57) ABSTRACT

Methods for producing a non-human meat products in a culture media containing egg yolk powder and culture media containing egg yolk powder are provided.

### CROSS-REFERENCE TO RELATED APPLICATIONS

This patent application claims the benefit of priority from U.S. Provisional Application Ser. No. 63/425,513, filed Nov. 15, 2022, teachings of which are incorporated by reference in their entirety.

### FIELD

This disclosure relates to methods for producing non-human meat products in a culture media containing egg yolk powder and culture media containing the egg yolk powder in concentrations effective for production of non-human meat products.

### BACKGROUND OF THE INVENTION

Meat products such as beef, pork, lamb, poultry, or fish are desirable products for food consumption.

The majority of meat products today produced from whole animals. This is a highly inefficient production method because a significant portion of all agriculturally produced grain is used for animal rather than human consumption. In addition, to produce one pound of beef, thousands of pounds of water are required for the animal to drink and to grow the livestock feed.

Further, current meat production methods are harmful to the environment. Rain forests are depleted at a rate of approximately 500 square feet of rain forest for every pound of beef to be grown. Likewise, modern techniques for fishing marine life have become so efficient that the oceans and lakes are over-fished. Species that were once common are now endangered or extinct.

Another problem with current meat production methods involves food contamination.

Cultured meat, sometimes called lab-grown, clean, or cultivated meat, is grown in a lab from a few animal cells. It is real meat, but it does not require animals to be slaughtered the way traditional meat does. To make lab-grown meat, scientists typically take stem cells from an animal. They bathe the cells in a liquid containing nutrients to help them duplicate and put them into a bioreactor to promote cell growth. Once the “unstructured” meat has developed, the next step is to make it a realistic meat product. Companies are trying to find the best way to produce burgers, nuggets, and other products from cultured meat. Some are using “scaffolding” made from soy protein, gelatin, or other sources to shape the lab-grown meat. Depending upon what kind of meat is being cultivated, this process should take 2 to 8 weeks.

In general terms, cultured cells require a sterile environment and a supply of nutrients for growth. In addition, the culture environment should be stable in terms of pH and temperature. Over the last 60 years, various defined basal media types have been developed and are now available commercially. Originally, balanced salt solutions were used to maintain contractility of mammalian heart tissue and Tyrode's salt solution was designed for use in work with primary mammalian cells. These founding formulations have since been modified and enriched with amino acids, vitamins, fatty acids and lipids so that modern culture media are suitable for supporting the growth of a wide range of cell types. The precise media formulations have often been derived by optimizing the concentrations of every constituent.

Serum is a complex mix of albumins, growth factors and growth inhibitors and is probably one of the most important components of cell culture medium. The most commonly used serum is fetal bovine serum (FBS). Other types of serum are available including newborn calf serum and horse serum. The quality, type and concentration of serum can affect the growth of cells.

In 1983, Fujii and Gospodarowicz disclosed supplementation of tissue culture medium with chicken egg yolk high in phosphatidyl choline to support the proliferation of low density bovine vascular and corneal endothelial cells and vascular smooth muscle cells maintained on basement-lamina-coated dishes (In Vitro 1983 19(11):811-817).

In 2000, Sasse et al. suggested replacement of fetal calf serum in cell cultures of a neuronal cell line with an egg yolk factor with cholecystokinin/gastrin-like immunoreactivity (ATLA 2000 28:815-831).

The egg yolk plasma, a translucent fraction of the egg yolk obtained by centrifugation has also been tested as a developmentally encouraging, cost-effective, biomaterial for salivary gland (SG) tissue engineering (Charbonneau and Tran Materials 2020, 13(21): 4807).

Various methods have been disclosed for use of cell culture in production of cultured meat.

For example, U.S. Pat. No. 6,835,390 discloses a method for differentiating myoblasts into specific myocytes or muscle cells by culturing the myoblasts in complete medium or in minimal media (e.g. complete medium less the fetal calf serum) supplemented with muscle specific growth or differentiation factors.

However, technologies such as this have not advanced sufficiently to support scalable, economically sustainable production as these methods utilize primary animal components such as animal tissues and serum, thereby negating the advantages of animal-autonomous meat production.

U.S. Pat. No. 10,920,196 discloses an in vitro, serum-free cultivation method for producing a cultured meat product for dietary consumption. However, this method requires modifying a porcine induced pluripotent stem cell line to comprise pluripotency genes POU5F1 and KLF4 with an inducible MYOD1 transcription factor to produce an inducible MYOD1-transcription-factor-modified porcine cell line. inducing myogenic differentiation of said modified cell line by exogenous regulation, and contacting the modified cell line with an activator of canonical WNT signaling, an inducer of MYoD1 expression, and an inhibitor of DNA methylation, wherein the inhibitor is 5-Aza-Cytidine or 5-Aza-2′-deoxycytidine, for the differentiated modified cell line to form myocytes and multinucleated myotubes which are then cultured to generate skeletal muscle fibers, thereby producing a cultured meat product for dietary consumption.

There exists a need for compositions and methods for use of these compositions to produce cultured non-human meat products which are fit for consumption that are, simpler, sustainable, more cost effective and more efficient than current compositions and methods for production.

### SUMMARY OF THE INVENTION

An aspect of the present invention relates to a method for producing a non-human meat product. The method comprises culturing non-human muscle cells ex vivo in culture media comprising a composition comprising egg yolk powder. The method further comprises seeding the non-human muscle cells onto a support structure and growing the non-human muscle cells to produce said non-human meat product.

In one nonlimiting embodiment, the egg yolk powder is optimized for solubility in culture media and/or included in an amount effective to promote growth of skeletal muscle cells.

Another aspect of this present invention relates to cell culture media comprising a composition comprising egg yolk powder.

In one nonlimiting embodiment, the egg yolk powder is optimized for solubility in culture media and/or included in an amount effective to promote growth of skeletal muscle cells.

### DETAILED DESCRIPTION OF THE INVENTION

Disclosed herein are methods for producing a non-human meat product suitable for consumption and cell culture compositions for use in the methods of production.

The methods and compositions comprise use of egg yolk powder or one or more proteins and/or lipids derived from egg yolk which are effective in growth and development of lean muscle tissue.

In one nonlimiting embodiment, the cell culture composition comprises egg yolk powder optimized for solubility in culture media. In one nonlimiting embodiment, the cell culture composition comprises FORTETROPIN. FORTETROPIN is a fertilized egg yolk derived product used as a dietary and nutritional supplement (MYOS RENS TECHNOLOGY INC. CORPORATION Cedar Knolls, NJ). A method for production of FORTETROPIN is disclosed in U.S. Pat. No. 8,815,320, teachings of which are herein incorporated by reference in their entirety.

In another nonlimiting embodiment, the cell culture composition comprises an avian follistatin such as described in U.S. Published Patent Application No. 2007/0275036 of U.S. application Ser. No. 16/151,601, the disclosure of which are incorporated herein by reference in their entirety and/or other proteins and/or lipids found in avian eggs and which are beneficial in growth and development of lean muscle tissue.

For purposes of the present invention, when the phrases “egg yolk powder” or “composition comprising egg yolk powder” are used, they are meant to be inclusive, but are not limited to, egg yolk powder, one or more proteins and/or lipids derived from egg yolk which are effective in growth and development of lean muscle tissue, egg yolk powder optimized for solubility in cell culture media, FORTETROPIN and/or avian follistatin.

In one nonlimiting embodiment, the egg yolk powder is used as a replacement to serum and/or other growth factors and additives typically used in cell culture media for cultured meat production.

In one nonlimiting embodiment, the egg yolk powder is used as a supplement to serum and/or other growth factors and additives typically used in cell culture media for cultured meat production, thereby decreasing the required amount of serum and/or other growth factors and additives.

In the methods of this invention, non-human muscle cells are cultured in culture media comprising a composition comprising egg yolk powder. In one nonlimiting embodiment, the non-human muscle cells are skeletal muscle cells.

In one nonlimiting embodiment, the non-human muscle cells are derived from an animal selected from the group consisting of mammals, birds, fishes, invertebrates, reptiles, and amphibians. Muscle cells may be derived from any non-human animals consumed by humans such as mammals (e.g. cattle, buffalo, pigs, sheep, deer, etc.), birds (e.g. chicken, ducks, ostrich, turkey, pheasant, etc.), fish (e.g. swordfish, salmon, tuna, sea bass, trout, catfish, etc.), invertebrates (e.g. lobster, crab, shrimp, clams, oysters, mussels, sea urchin, etc.), reptiles (e.g. snake, alligator, turtle, etc.), and amphibians (e.g. frog legs).

In one nonlimiting embodiment, the non-human muscle cells are differentiated from a pluripotent or totipotent stem cell line that can proliferate indefinitely. Both embryonic stem cells and adult stem cells having a minimal self-renewal capacity may be suitable.

Preferably, muscle cells are derived from pluri-potent embryonic mesenchymal stem cells that give rise to muscle cells, fat cells, bone cells, and cartilage cells. The muscle cells may also be derived from toti-potent embryonic stem cells such as cells from the blastocyst stage, fertilized eggs, placenta, or umbilical cords of these animals.

The methods further comprise seeding the non-human muscle cells onto a support structure and growing the non-human muscle cells to produce said non-human meat product, wherein said non-human meat product is suitable for consumption.

The methods may further comprise seeding non-human fat cells and/or non-human cartilage cells onto the support structure so that the non-human fat cells and/or non-human cartilage cells grow in conjunction with the non-human muscle cells to produce a non-human meat product.

The methods may further comprising seeding non-human embryonic fibroblasts and/or non-human endothelial cells with the non-human muscle cells.

Non-human fat cells, cartilage cells, embryonic fibroblasts and/or endothelial cells may be derived from an animal selected from the group consisting of mammals, birds, fishes, invertebrates, reptiles, and amphibians and/or differentiated from pluripotent or totipotent non-human stem cells.

Cells may be grown in culture media comprising egg yolk powder into muscle tissues that are attached to a support structure such as a two or three-dimensional scaffold or support structure. The muscle cells may be grown on the two dimensional support structure such as a petri-dish forming several layers of cells that may be peeled and processed for consumption. Other examples of two dimensional support structures may include porous membranes that allow for diffusion of nutrients from culture media comprising egg yolk powder on one side of the membrane to the other side where the cells are attached. In this type of culture conditions, additional layers of cells may be achieved by exposing the cells to culture media comprising egg yolk powder from both sides of the membrane, i.e., cells received nutrients through diffusion from one side of the membrane and also from the culture media comprising egg yolk powder covering the cells growing on the membrane.

Cells may also be grown on, around, or inside a three-dimensional support structure. The support structure may be sculpted into different sizes, shapes, and forms, as desired, to provide the shape and form for the muscle cells to grow and resemble different types of muscle tissues such as steak, tenderloin, shank, chicken breast, drumstick, lamb chops, fish fillet, lobster tail, etc. The support structure may be made from natural or synthetic biomaterials that are preferably non-toxic so that they may not be harmful if ingested. Natural biomaterials may include, for example, collagen, fibronectin, laminin, or other extracellular matrices. Synthetic biomaterials may include, for example, hydroxyapatite, alginate, polyglycolic acid, polylactic acid, or their copolymers. The support structure may be formed as a solid or semisolid support.

To provide for optimal cell and tissue growth, the support structure, preferably, has high porosity to provide maximal surface area for cell attachment. A three-dimensional support structure may also be molded to include a branched vascular network providing for delivery of nutrients into and shuttling out of metabolites from the cells at the inner mass of the meat product. In this embodiment, the branch vascular network may be edible by using non-toxic natural or synthetic biomaterials as mentioned above. Furthermore, the support structure may also include adhesion peptides, cell adhesion molecules, or other growth factors covalently or non-covalently associated with the support structure. Examples of the peptides include sequences such as Arg-Gly-Asp or Arg-Glu-Asp-Val. Niklason, L., et. al., Advances in Tissue Engineering of Blood Vessels and Other Tissues, Transplant Immunology, 5(4):303-306 (1997).

In one nonlimiting embodiment, culturing is performed on a large scale basis in in large bioreactors which provide adequate solid surface space required by the stem cells and/or skeletal muscle cells to generate sufficient numbers of muscle cells.

In one nonlimiting embodiment, the bioreactor maintains low shear and uniform perfusion at large volumes to promote the growth of tissue cultures which accurately resemble native tissue architecture and provides an environment which allows for increased culture volumes.

In one nonlimiting embodiment, the bioreactor is a rotating wall vessel bioreactor. These bioreactors provide for laminar flow of the medium comprising egg yolk powder by rotating the cylindrical wall at a speed that balances centrifugal force, drag force and gravitational force, leaving the support structure seeded with cells submerged in the medium in a perpetual free fall state that improves diffusion with high mass transfer rates at minimal levels of shear stress, thereby producing three dimensional tissues with structures very similar to those in vivo.

In one nonlimiting embodiment, the bioreactor is a direct perfusion bioreactor useful for scaffold based myocyte cultivation in that it allows flow of medium comprising egg yolk powder through a porous scaffold with gas exchange taking place in an external fluid loop. In addition to offering high mass transfer, this bioreactor also offers significant shear stress, so determining an appropriate flow rate is essential. Direct perfusion bioreactors are also used for high-density, uniform myocyte cell seeding.

The methods and compositions disclosed herein provide provides a simpler, more sustainable, more cost effective and more efficient means for production of cultured meat products.

1. A method for producing a non-human meat product suitable for consumption, said method comprising culturing non-human muscle cells ex vivo in culture media comprising a composition comprising egg yolk powder.

2. The method of claim 1 further comprising:
seeding the non-human muscle cells onto a support structure; and
growing the non-human muscle cells to produce said non-human meat product, wherein said non-human meat product is suitable for consumption.

3. The method of claim 1 wherein the composition comprises egg yolk powder optimized for solubility in culture media.

4. The method of claim 1 wherein the composition comprises egg yolk powder in an amount sufficient to promote growth of skeletal muscle cells.

5. The method of claim 1 wherein the composition comprises a fertilized egg yolk derived product.

6. The method of claim 1 wherein the egg yolk powder is derived from fertilized avian eggs.

7. The method of claim 1 wherein the egg yolk powder is produced via high pressure pasteurization (HPP) and freeze drying of at least a portion of an egg.

8. The method of claim 1 wherein the egg yolk powder is produced via spray drying at least a portion of an egg.

9. The method of claim 1 wherein the composition is FORTETROPIN.

10. The method in claim 1 wherein the non-human muscle cells are skeletal muscle cells.

11. The method in claim 1 wherein the non-human muscle cells are derived from pluripotent or totipotent non-human stem cells.

12. Cell culture media comprising a composition comprising egg yolk powder.

13. The cell culture media of claim 12 wherein the egg yolk powder is optimized for solubility in culture media.

14. The cell culture media of claim 12 wherein the egg yolk powder in included an amount sufficient to promote growth of skeletal muscle cells.

15. The cell culture media of claim 12 wherein the composition comprises a fertilized egg yolk derived product.

16. The cell culture media of claim 12 wherein the egg yolk powder is derived from fertilized avian eggs.

17. The cell culture media of claim 12 wherein the egg yolk powder is produced via high pressure pasteurization (HPP) and freeze drying of at least a portion of an egg.

18. The cell culture media of claim 12 wherein the egg yolk powder is produced via spray drying at least a portion of an egg.

19. The cell culture media of claim 12 wherein the composition is FORTETROPIN.
